(19) United States
(12) Patent Application Publication
(10) Pub. No.: US 2024/0208901 A1
(43) Pub. Date: Jun. 27, 2024

(54) Deuterated 3,3'-Diselenodipropionic Acid (DSePA) and Its Use as an Anticancer or Radioprotective Agent

(71) Applicant: Secretary, Department of Atomic Energy, Mumbai (IN)

(72) Inventors: Amit Kunwar, Mumbai (IN); Manoj K. Pal, Mumbai (IN); Vishwa Vipulkumar Gandhi, Gujarat (IN); Sandip Dey, Mumbai (IN); Ram Pada Das, Mumbai (IN); Beena Gobind Singh, Mumbai (IN); Amey P. Wadawale, Mumbai (IN)

(21) Appl. No.: 18/087,427

(22) Filed: Dec. 22, 2022

(51) Int. Cl. C07C 391/00

(52) U.S. Cl. CPC C07C 391/00; C07B 2200/05

(57) ABSTRACT

The present invention applies to a deuterated derivative of 3,3′-diselenodipropionic acid (D-DSePA), an organo-selenium compound and method of synthesis thereof. The invention also provides the method including D-DSePA for the inhibition of the proliferation of human non-small cell lung carcinoma (NSCLC) cells. This compound inhibits the proliferation of A549, H1975 and H3122 cells of NSCLC origin at a much lower concentration than that needed to inhibit the growth of non-cancerous lung fibroblast cells (WI-38 and WI-26 VA4). Further, D-DSePA exhibits additive effect in A549 lung cancer cells while protecting non-cancerous WI38 cells from radiotoxicity.

### BACKGROUND OF THE INVENTION

### Field of Invention

The present invention applies to a deuterated derivative of 3,3′-diselenodipropionic acid (D-DScPA), an organo-selenium compound and method of synthesis thereof. The invention also provides the method comprising D-DSePA for the inhibition of the proliferation of human non-small cell lung carcinoma (NSCLC) cells. In brief, said compound inhibits the proliferation of A549, H1975 and H3122 cells of NSCLC origin at a much lower concentration than that needed to inhibit the growth of non-cancerous lung fibroblast cells (WI-38 and WI-26 VA4). Further, D-DScPA exhibits additive effect in A549 lung cancer cells while protecting non-cancerous WI38 cells from radiotoxicity.

### Description of Related Art

Non-small cell lung carcinoma (NSCLC) is one of the most aggressive cancer types in terms of incidence, resistance towards chemotherapy and radiotherapy and is ranked within the top five in terms of mortality. The organoselenium compound, 3,3′-diselenodipropionic acid (DScPA) has previously been reported for various pharmacological activities including the anticancer activity. It has been shown to specifically inhibit the growth of human non-small cell lung carcinoma (NSCLC) cells by modulating intracellular redox state towards reduction. Further, DScPA has all the attributes required in a candidate drug molecule including scalable synthetic route, oral bioavailability, preferential pulmonary biodistribution, and selectivity for lung tumor compared to normal lung tissue. However, increasing the therapeutic index of DSePA or widening the margin between its beneficial effect in tumor cells and toxicity in normal cells will further enhance its appeal for possible clinical translation in future. DSePA being an organodiselenide (R-Se-Se-R) can react with metabolic enzymes or oxidizing agent to form seleninic acid (RSeO₂H) and other degraded by-products which can cause toxicity limiting its therapeutic index. Over the years, researchers have established that the toxicity of selenium compounds is governed by their rate of metabolism. For example, organic selenium compounds undergo slower metabolism as compared to inorganic selenium compounds and therefore are considered to be much safer as compared to the latter group of compounds. In this context, deuteration of the drug molecule is the most commonly used strategy for increasing the metabolic stability or slowing the reactivity. In deuterated compounds, the hydrogen (H) atom is replaced by its heavier isotope deuterium (D) which is not radioactive. The two-fold increase in the mass of D compared with H, results in the shorter bond length of the C-D bond. Accordingly, more energy is required to break the C-D bond and as a result deuterated compounds show lower reactivity for oxidative/reductive processes. To the best of our knowledge, there is no report on the influence of deuteration on the toxicological and efficacy parameters of any pharmacologically active organoselenium compound till date. Thus, inventors hypothesized that D-DSePA might be effective in improving its therapeutic index in terms of the differential toxicity between normal and cancer cells. In order to address the above said hypothesis, the inventors evaluated the toxicity of D-DSCPA in human NSCLC cells such as A549, H1975 and H3122 cells and in non-cancerous human lung fibroblast cells like WI-38 and WI-26 VA4. Further, treatment of NSCLC requires multimodal approach involving combination of chemotherapy and radiotherapy (exposure of ionizing radiation like X ray, γ-ray). Therefore, it is also important to understand the anticancer efficacy of D-DSePA in combination to exposure of ionizing radiation. Accordingly, the inventors evaluated the toxicity of D-DSePA in human NSCLC cells (A549) with or without gamma (γ)-irradiation. The results were further compared with the toxicity of D-DSePA in non-cancerous human lung fibroblast cells (W138) with or without gamma (γ)-irradiation.

DSePA has all the attributes required in a candidate drug molecule including scalable synthetic route, oral bioavailability, preferential pulmonary biodistribution, and selectivity for lung tumor compared to normal lung tissue for the treatment of NSCLC. However, increasing the therapeutic index of DSePA or widening the margin between its beneficial effect in tumor cells and toxicity in non-cancerous cells is expected to further enhance its appeal for possible clinical translation in future. Accordingly, the present invention sought to reduce the toxicity of DSePA in non-cancerous cells without compromising its efficacy in lung cancer cells by synthesizing a new derivative, D-DSePA. Additionally, the beneficial effects in terms of radioprotection by D-DSePA was also evaluated in non-cancerous cells.

Previous publications of the applicant have established that DSePA is a potent anticancer and radioprotective agent with a narrow therapeutic index. The investigations on the mechanisms of actions have suggested that the pharmacological activity of DSePA is mainly due to its metabolic assimilation into selenoproteins. However, it is also well established that the metabolic byproducts of aliphatic organoselenium compounds contribute systemic toxicity. Therefore, developing a methodology or strategy to temporally regulate the metabolism of aliphatic organoselenium compounds without compromising their pharmacological efficacy is a challenge. In this regard, it was conceived to synthesize the deuterated derivative of DSePA as deuteration is a well-known strategy to slow down the metabolism of drug molecule.

The subject invention D-DSePA is applied for cancer treatment and in reducing radiotherapy side effects.

### Objectives of the Invention

The principal object of the present invention is to develop a method for synthesizing D-DSePA.

The other objective of the present invention is to evaluate the toxicity of D-DSePA in human lung cells of cancerous (NSCLC) and non-cancerous (fibroblast) origins in combination with or without gamma (γ)-irradiation.

### SUMMARY OF THE INVENTION

Accordingly, the present invention discloses a method of synthesis of a organodiselenide derivative 3,3′-diselenodipropionic acid-D8 (D-DSePA). The method comprises of reacting freshly prepared sodium selenide (Na₂Se₂) and 3-bromopropionic acid-D₄ in D₂O solvent. The precursors used for the synthesis are selenium powder, sodium borohydride and 3-bromopropionic acid-D₄. The reaction was carried out in deuterated water (D₂O).

In another embodiment, it discloses a method of inhibiting the growth/proliferation of non-small cell lung cancer (NSCLC) with higher therapeutic index, comprising treating/incubating cellular models of NSCLC and non-cancerous lung fibroblast with deuterated 3,3′-diselenodipropionic acid (D-DSePA) in vitro. The NSCLC is a A549 or H1975 or H3122 cell type and the non-cancerous lung fibroblast is a WI-38 or WI-26 VA4 cell type.

In yet another embodiment, the present invention discloses a method of inhibiting the growth/proliferation of non-small cell lung cancer (NSCLC) comprising treating/incubating cellular models of NSCLC with γ-irradiation and deuterated 3,3′-diselenodipropionic acid (D-DSePA) in vitro. The NSCLC is a A549 cell type. Further, the cells are γ-irradiated in the absorbed dose range of 2-8 Gy. Here the cells are treated with D-DSePA at a concentration of 0.5 μM immediately after radiation exposure.

In yet another embodiment, the present invention discloses a method of radioprotection by treating/incubating non-cancerous cells with deuterated 3,3′-diselenodipropionic acid (D-DSePA) in vitro. Here the non-cancerous cell is WI-38 cell type. The cells are irradiated in the absorbed dose range of 2-12 Gy. and are treated with D-DSePA at a concentration of 0.5 μM. The cells are treated with D-DSePA at 24 hours prior to radiation exposure.

### BRIEF DESCRIPTION OF THE DRAWINGS

FIG. 1 illustrates a scheme showing chemical reaction involved in the synthesis of D-DSePA.

FIG. 2 is a flow diagram showing various steps involved in the synthesis of D-DSePA.

FIG. 3 illustrates FT-IR spectrum (in KBr) of D-DSePA.

FIG. 4 illustrates ¹H NMR spectrum (300 MHZ, DMSO-D₆) of D-DSePA.

FIG. 5 illustrates ¹³C{¹H} NMR spectrum (75.5 MHz, methanol-D₄) of D-DSePA

FIG. 6 illustrates ⁷⁷Se{¹H} NMR spectrum (57.2 MHZ, methanol-D₄) of D-DSePA

FIG. 7 illustrates GC-MS spectrum of D-DSePA.

FIG. 8 illustrates Molecular structure of D-DSePA ellipsoids drawn at 35% probability.

FIG. 9 shows the cytotoxicity of DSePA and D-DSePA in (A) A549 (B) H1975 (C) H3122 (D) WI-38 and (E) WI-26 SV4A. Cells like A549, H1975 and H3122 are the representative of human NSCLC. Cells like WI-38 and WI-26 SV4A are the representative of human lung fibroblast. The cells were treated with DSePA/D-DSePA for 48 hours prior to MTT assay. The experiments were done in triplicates. The results are presented as mean±SEM (n=3).

FIG. 10 shows the mean therapeutic index of DSePA and D-DSePA in NSCLC cells as compared normal lung fibroblast cells. The result is presented as mean±SEM (n=6). #<0.05 as compared to DSePA group by T Test.

FIG. 11 shows comparison of the toxicity of DScPA and D-DSePA in A549 and WI-38 cells by clonogenic assay. (A) Representative images show the toxicity of DScPA and D-DSePA in terms of reduction in size and number of colonies in A549 and WI-38 cells by clonogenic assay at an identical treatment concentration of 1 μM. (B) Plot shows the effect of DSePA/D-DSePA (0.1- 2 μM) on the clonogenic potential of A459 cells in terms of survival fraction. (C) Plot shows the effect of DSePA/D-DScPA (1- 500 μM) on the clonogenic potential of WI-38 cells in terms of survival fraction. The survival plots were used to estimate the IC₅₀ of DScPA/D-DScPA for inhibiting the growth of A549/WI-38. The results are presented as mean±SEM (n=3).

FIG. 12 shows comparison of the radio-modulating activity of DSePA and D-DScPA in A549 and WI-38 cells by clonogenic assay. (A) Representative images show effect of the combinatorial treatment of the varying absorbed (2-8 Gy) doses of γ-radiation and DSePA (0.5 μM) and on the viability A549 cells in terms of reduction in size and number of colonies by clonogenic assay. (B) Plot shows the effect of the combinatorial treatment on the clonogenic potential of A459 cells in terms of survival fraction. (C) Representative images show the effect of the pre-treatment (24 hour) of DScPA/D-DScPA (0.5 μM) against the γ-radiation (2-12 Gy)-induced cytotoxicity in WI-38 cells by clonogenic assay. (B) Plot shows the protective effect of DSePA/D-DSePA against γ-radiation (2-12 Gy) mediated decrease in the survival fraction of WI-38 cells. The results are presented as mean±SEM (n=3).

### DETAILED DESCRIPTION OF THE INVENTION

While the embodiments of the disclosure are subject to various modifications and alternative forms, specific embodiment thereof have been shown by way of example in the figures and will be described below. It should be understood, however, that it is not intended to limit the disclosure to the particular forms disclosed, but on the contrary, the disclosure is to cover all modifications, equivalents, and alternative falling within the scope of the disclosure.

The figures illustrate only those specific details that are pertinent to understand the embodiments of the present disclosure, so as not to obscure the disclosure with details that will be clear to those of ordinary skill in the art having benefit of the description herein.

3,3′-Disclenodipropionic acid-D₈ (D-DSePA, chemical structure shown in FIG. 1) was synthesized by reacting freshly prepared sodium selenide (Na₂Se₂) and 3-bromopropionic acid-D₄ in D₂O solvent with 42% yield. The synthesized D-DSePA was purified and characterized by NMR spectroscopy and mass spectrometry. The molecular structure of D-DSePA has been established by single-crystal X-ray diffraction analysis. The treatment of D-DSePA and un-deuterated DSePA inhibited the growth of human NSCLC cells such as A549, H1975 and H3122 with half maximum concentration (IC₅₀) in the range of 5-50 μM by MTT assay. However, D-DSePA was significantly lesser toxic (˜4 to 5 times higher IC₅₀) as compared to un-deuterated DSePA in non-cancerous human lung fibroblast cells (WI-38 & WI-26 VA4) confirming higher therapeutic index of D-DScPA. Further, D-DSePA did not protect A549 NSCLC cells from radiation kill justifying its application for combinatorial therapy along with radiotherapy. Finally, at identical treatment concentration, D-DSePA offered better radioprotection to non-cancerous WI38 cells with a dose modification factor (DMF) of 1.16±0.05 as compared to un-deuterated DScPA with DMF of 1.12±0.03.

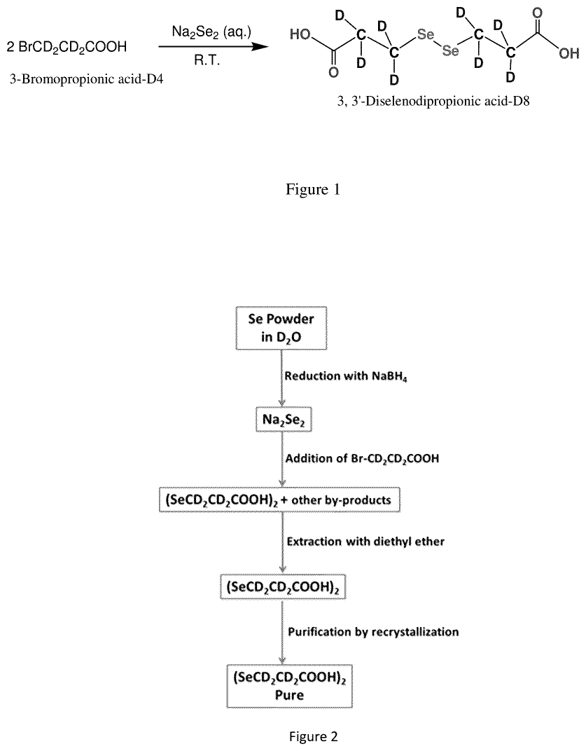

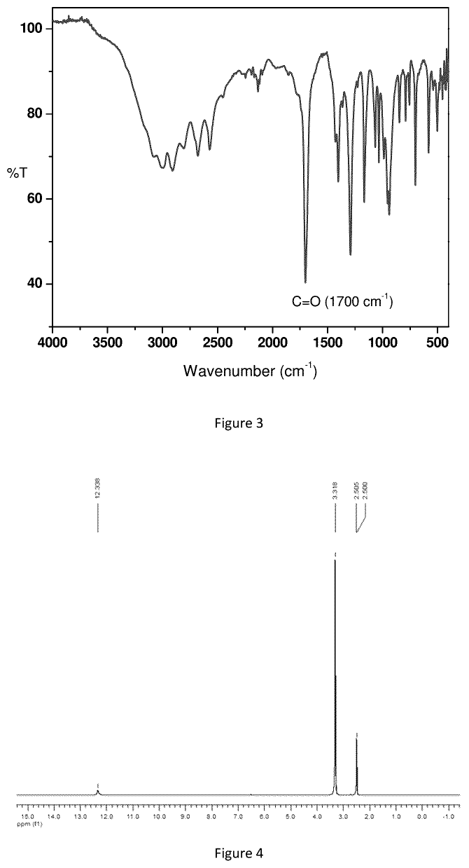

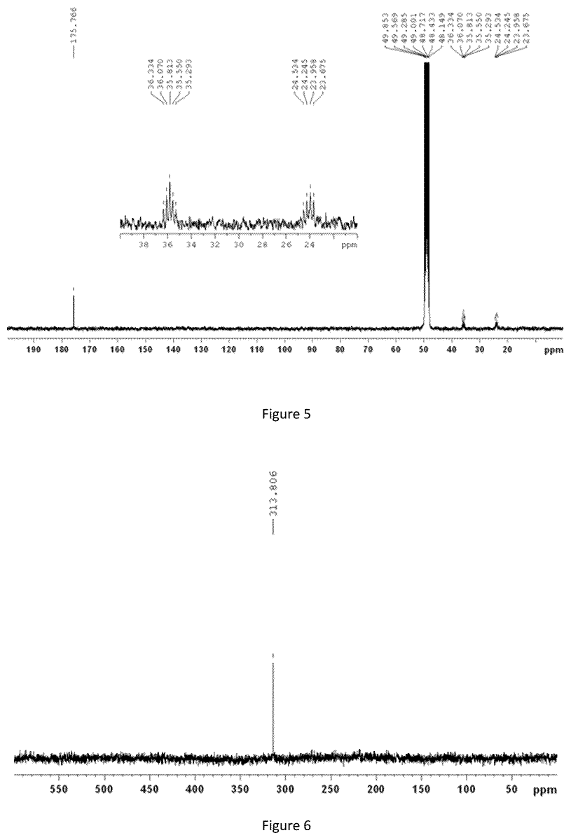

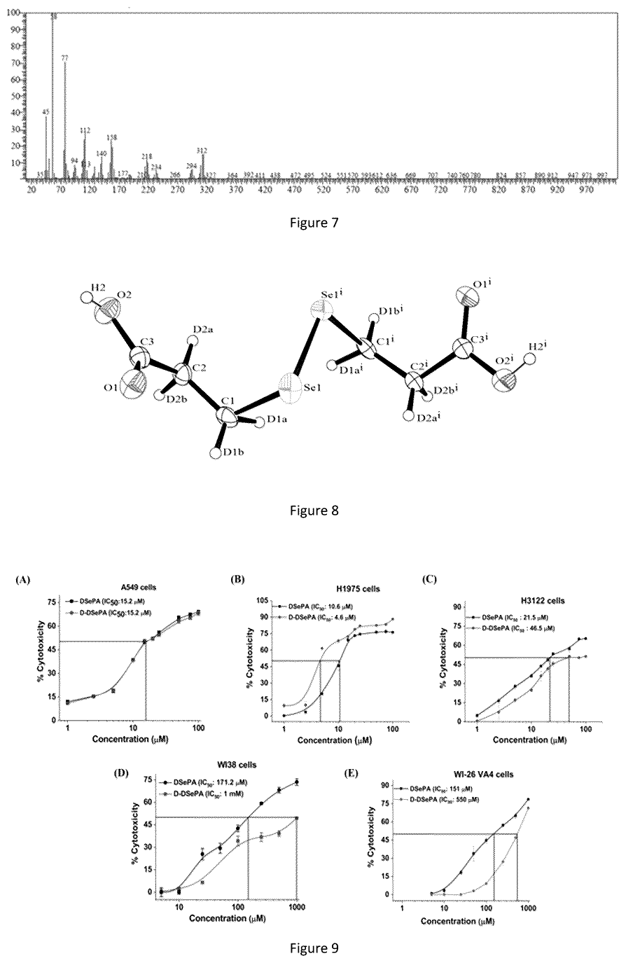

The 3,3′-diselenodipropionic acid-D₈ (D-DSePA) was synthesized by nucleophilic substitution reaction from Na₂Se₂ and 3-bromopropionic acid-D₄ in D₂O solvent (FIG. 1 and FIG. 2). The recrystallized D-DSePA was characterized by elemental analysis, IR, NMR (¹H, ¹³C{¹H} and ⁷⁷Sc{¹H}) spectroscopy and mass spectrometry. The IR spectrum showed characteristic strong absorption band at 1700 cm⁻¹, attributed to C═O stretching vibration of the carboxyl carbon (FIG. 3). The ¹H NMR spectrum of D-DSePA in methanol-D₄ and acetone-D₆ displayed only the solvent residual peak and the water peak, thus confirms the purity of the synthesized organic compound. The COOH peak was not detected due to the intermolecular exchange with solvent and water. Interestingly, the ¹H NMR spectrum of D-DSePA in dmso-D₆ containing less amount water, showed an additional broad singlet at δ 12.34 ppm for the COOH group (FIG. 4). The ¹³C{¹H} NMR spectrum displayed two pentate peaks at δ 23.9 and 35.8 ppm attributed to SeCD₂ and SeCD₂CD₂ carbons, respectively with ¹J_{D-C} coupling constant of ˜19.4-21.7 Hz (FIG. 5). The sharp peaks associated with ²D-¹³C coupling indicate no exchange of protons, and confirms the intact “CD₂CD₂” moicty of D-DSePA in solution. The sharp singlet at δ 175.8 ppm has been assigned to carboxylic group carbon. The ⁷⁷Se{¹H} NMR spectrum in methanol-D₄ showed a singlet at δ 314 ppm indicating the symmetric nature of diselenide linkage (FIG. 6). It is close to the chemical shift of non-deuterated DSePA i.c., 316 ppm in ⁷⁷Se{¹H} NMR spectrum. The mass analysis of D-DScPA was performed by direct ionization technique (FIG. 7). The mass spectrum clearly showed the peak at m/z 312 corresponding to ([M−2H]⁺, 16%) and confirms the formation of (ScCD₂CD₂COOH)₂. The other prominent peaks were observed at m/z 294, 234, 218, 158, 140, 112, 94, 77, 58 and 45, which have been attributed to the fragmented ions ([M−(H+D+OH)]⁺, 6%), ([M−(COOH+O₂H+D)]⁺, 5%), ([M−(2COOH+3D)]+, 12%), ([SeCD₂CD₂COOH+H]⁺, 23%), ([SeCD₂CD₂CO]+, 15%), ([ScCD2CD2]+, 28%), ([ScCD]+, 8%), ([CD2CD2COOH]+, 70%), ([CD2CDCO]+, 100%), ([COOH]⁺, 38%) respectively. Similarly, the mass spectrum of un-deuterated DSePA was analyzed for comparison. The observed peak at m/z 306 has been assigned to [M]⁺ for (ScCH₂CH₂COOH)₂. The structure of D-DSePA was unambiguously determined by X-ray crystallography (FIG. 8, Table-1 and Table-2). The asymmetric unit cell contains only a half of the molecule, the entire molecule is generated via an inversion center that is located at the mid-point between the two Se atoms. The crystal packing displays the molecules are stacked along the b axis with a distance of 4.56 Å between the planes containing diselenide bond. The molecule can adopt the helical conformations due to the tetrahedral C or Se centers, determined by gauche —C—C—Se—Se— or —C—Se—Se—C— torsion angles c.a. −58° or −81°, respectively. The —C—C—Se—Se— torsion angles in three acyclic diselenides (Code: CIMCEH, GIWXAO and QIHNUQ) containing the group “(—CH₂—CH₂—Sc—)₂” retrieved from CSD (version 2021.3.0) falls in the range of 60-98°. The Se—C and Se—Se bond distances of about 1.95 and 2.31 Å, respectively are comparable with the reported values in (SeCH₂COOH)₂ and (SeCH₂CHNH₂COOH)₂·2HCl. The C-D bond distances of about 0.97 Å match well with the reported C-D bond distances of S-based heterocyclic compound. Two free carboxylic acid groups are involved in intermolecular H-bonding forming a carboxyl dimer synthon which are described by graph set R₂²(8). One dimensional zigzag chain is formed from repetition of dimer pattern through hydrogen bonding, as observed in dicarboxylic acid, such as terephthalic acid. The 8-membered ring of carboxyl dimer deviates from planarity and forms puckered structure due to the deviation of donor-H-acceptor angle (D-H . . . A) from the linearity such as O2-H2 . . . O1˜161.8°. The donor-acceptor (O1 . . . O2: 2.64 Å) and H . . . acceptor (H2 . . . O1: 1.85 Å) distances are comparatively shortened, possibly due to stronger H-bond interactions. Interestingly, van der Waals interactions between two infinite chains in the crystal lattice exist through one deuterium of one chain and oxygen atom of another chain (D1b . . . O2: 2.72 Å).

TABLE 1
Selected bond lengths (Å) and angles (°) for D-DSePA

| | | | |
|---|---|---|---|
| Se(1)—C(1) | 1.953(11) | C(1)—C(2) | 1.486(15) |
| Se(1)—Se(1)ⁱ | 2.307(2) | C(1)-D(1A) | 0.97(2) |
| C(3)—O(1) | 1.230(12) | C(3)—O(2) | 1.323(12) |
| C(1)—Se(1)—Se(1)ⁱ | 102.1(3) | C(2)—C(1)—Se(1) | 114.4(7) |
| C(2)—C(1)-D(1A) | 121(5) | Se(1)—C(1)-D(1A) | 82(7) |
| C(2)—C(1)-D(1B) | 118(7) | Se(1)—C(1)-D(1B) | 100(7) |

TABLE 2
Crystallographic and structure refinement data for D-DSePA

| Compounds | D-DSePA |
|---|---|
| Chemical Formula | C₆H₂D₈O₄Se₂ |
| Formula weight | 312.08 |
| Crystal Size (mm³) | 0.10 × 0.05 × 0.05 |
| Diffractometer | Rigaku-Oxford make XtaLAB Synergy, |
| | Dualflex X-ray diffractometer |
| T/K | 298(2) |
| λ/Å | MoKα (λ = 0.71073 Å) |
| Crystal system | Monoclinic |
| Space group | C 2/c |
| a/Å | 5.5158(6) |
| b/Å | 9.1291(9) |
| c/Å | 19.1868(19) |
| α/° | 90 |
| β/° | 94.815(9) |
| γ/° | 90 |
| V/Å³ | 962.73(17) |
| ρ_{calc}/g cm⁻³ | 2.153 |
| Z | 2 |
| μ/mm⁻¹ | 7.655 |
| Reflection collected/unique | 3830/1202 |
| Data/restraints/parameters | 1202/5/69 |
| Final R₁, wR₂ indices | R1 = 0.0917, wR2 = 0.2470 |
| R₁, wR₂ (all data) | R1 = 0.1183, wR2 = 0.2572 |
| Largest diff. peak & hole [eÅ⁻³] | 2.745 and −0.703 |

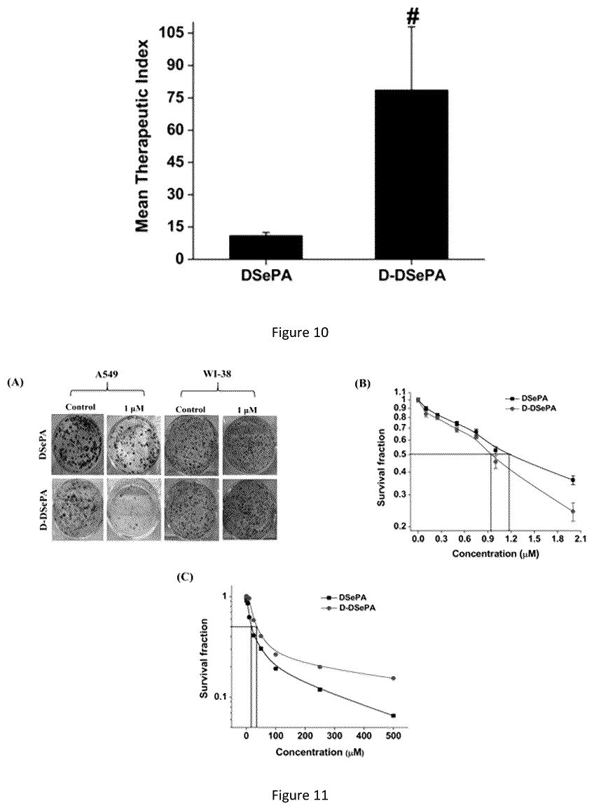

Notably, un-deuterated DSePA has been shown to exhibit anticancer activity in cellular models of NSCLC. Therefore, D-DSePA was compared with un-deuterated DSePA for its anticancer activity in A549, H1975 and H3122 cell lines which are representative of NSCLC. For this, above cells were treated with the varying concentrations (0.1-100 μM) of DSePA/D-DSePA for 48 h and the cell viability was determined by MTT assay (FIG. 9A-C). The plot of percent (%) cytotoxicity (100-% viability) versus the concentration of DSePA/D-DSePA was used to estimate the half maximum concentration (IC₅₀) of above compounds to inhibit the proliferation of cells. The results indicated that DSePA and D-DSePA inhibited the cell proliferation with the IC₅₀ values of 15.2±1.0 μM and 15.2±0.9 μM respectively in A549 cells, 10.6±1.1 μM and 4.6±0.7 μM respectively in H1975 cells and 21.5±1.2 μM and 46.5±2.9 μM respectively in H3122 cells. Similarly, the toxicity of DSePA/D-DSePA was studied in non-cancerous lung fibroblast cells, WI-38 and WI-26 VA4 in the concentration range of 0.1 to 1000 μM at 48 h post treatment by MTT assay (FIG. 9D and 9E). The IC₅₀ of DSePA and D-DSePA for inhibiting the cell proliferation was 171.2±2.2 μM and 1000±6.3 μM respectively in WI38 cells and 151±3.3 μM and 550±10.2 μM respectively in WI-26 VA4 cells. Employing these values, the therapeutic index (IC₅₀ in normal lung cell/IC₅₀ in lung cancer cell) of DSePA and D-DSePA in different pairs of normal versus cancerous cells (viz., A549/WI-38, A549/WI-26 VA4, H1975/WI-38, H1975/WI-26 VA4, H3122/WI-38 and H3122/WI-26 VA4) was calculated (Table-3). As per the results shown in table-3, values of therapeutic index varied depending on cell lines however, D-DSePA consistently exhibited higher therapeutic index as compared to DSePA in all the studied NSCLC cells. The average therapeutic index of DSePA and D-DSePA estimated from above study was 11.05±1.5 and 78.7±29.2 respectively (FIG. 10).

TABLE 3
Therapeutic index of DSePA and D-DSePA in different lung cancer
cell lines (A549, H1975 and H3122) versus normal lung cells
(WI-38 and WI-26 VA4) as determined by MTT assay is presented.
The cytotoxicity was estimated at 48 h post-treatment.

| Therapeutic Index (IC₅₀ in | | |
|---|---|---|
| normal cell / IC₅₀ in cancer | | |
| cells) | DSePA | D-DSePA |
| A549/WI-38 | 11.2 ± 0.6 | 65.7 ± 3.3 |
| A549/WI-26 VA4 | 9.9 ± 0.5 | 36.2 ± 1.9 |
| H1975/WI-38 | 16.1 ± 0.8 | 217.4 ± 10.9 |
| H1975/WI-26 VA4 | 14.2 ± 0.7 | 119.6 ± 5.9 |
| H3122/WI-38 | 7.9 ± 0.4 | 21.5 ± 1.0 |
| H3122/WI-26 VA4 | 7.0 ± 0.4 | 11.8 ± 0.6 |

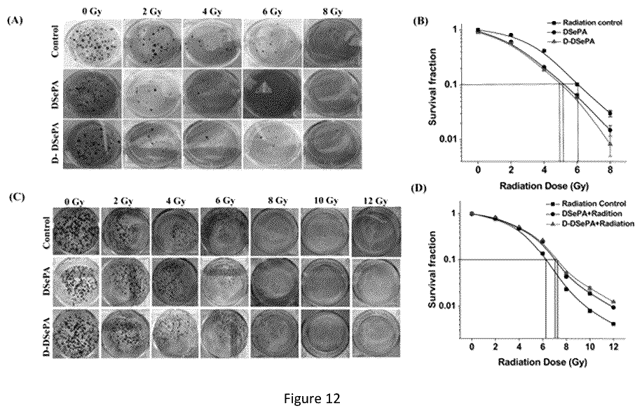

Further, clonogenic assay is considered as the gold standard method for evaluating the anticancer activity of any compound and is also a good indicator of the in vivo efficacy of any anticancer compound. Therefore, the therapeutic index of DSePA and D-DSePA in A549/WI-38 cells was also validated by employing the clonogenic assay. For this, A549/WI-38 cells were treated with the varying concentrations (0.1-2 μM)/(1-500 μM) of DSePA/D-DSePA for seven days and the survival fraction was calculated by counting the number of surviving clones (FIG. 11A). The plot of survival fraction verses the concentration of DScPA/D-DSCPA was used to estimate the IC₅₀ of above compounds to inhibit the growth of A549/WI-38 cells. The IC₅₀ values of DScPA and D-DScPA estimated from this analysis was 1.2±0.01 μM and 0.9±0.03 μM respectively in A549 cells and 17.9±0.01 μM and 35.5±0.02 μM respectively in WI-38 cells (FIG. 11B & 11C). This clearly confirmed that deuteration of DScPA reduced its toxicity in normal cells without compromising its efficacy in cancer cells. Since anticancer agents are intended to be used in combination with radiation, DSePA/D-DSePA at a treatment concentration of 0.5 μM (dose close to IC₅₀ as determined by clonogenic assay) was combined with varying absorbed doses (2-8 Gy) of γ-irradiation and the effect of combinatorial treatment on the viability of A549 cells was determined by clonogenic assay (FIG. 12A & 12B). For this, A549 cells exposed to the varying absorbed doses (2-8 Gy) of γ-radiation were immediately treated with DScPA/D-DScPA and cultured for seven days prior to clonogenic assay. The radiation alone group showed reduction in survival fraction in a dose dependent manner from which the decimal reduction dose (D₁₀, absorbed dose of the radiation that reduces survival fraction by one log cycle) estimated was 5 Gy (FIG. 12B). The combinatorial treatment showed lower survival fraction as compared to the drug (DSCPA/D-DScPA) alone or radiation alone group (FIG. 12B). The D₁₀ value of the combinatorial treatment involving DScPA estimated was 4.5 Gy (FIG. 12B). Similarly, D₁₀ value of the combinatorial treatment involving D-DSePA estimated was 4 Gy. From these values, the dose modification factor (DMF or the ratio of the D₁₀ of combinatorial treatment to that of D₁₀ of radiation) of DScPA and D-DScPA estimated was 0.87±0.01 and 0.83±0.02 respectively. This confirmed that D-DScPA did not protect lung cancer cells from radiation kill instated showed radio-sensitization. While the objective of radiotherapy is to achieve tumor control, it is also known to cause normal tissue toxicity. Previously, DSePA has been reported to protect normal cells from lethal effects of γ-radiation. Thus radio-protective activity of D-DSePA was compared with DScPA in terms of their abilities to protect non-cancerous lung fibroblast (WI-38) cells against γ-irradiation induced cell death. For this, WI-38 cells were pretreated with DSePA/D-DScPA for 24 h at an identical concentration of 0.5 μM (close to IC₅₀ required to inhibit growth of A549 lung cancer cells by clonogenic assay) and then exposed to varying doses (2-12 Gy) of γ-irradiation. The survival of cells against radiation exposure was estimated by clonogenic assay and the dose response curve was prepared in the presence and absence of drug (DSePA/D-DScPA) (FIG. 12C & 12D). By using this dose response curve, the dose modification factor (DMF or the ratio of the D₁₀ of radiation plus drug and D₁₀ of radiation alone) of DSePA and D-DSePA estimated was 1.12±0.03 and 1.16±0.05 respectively. This confirmed that D-DSePA exhibited radioprotective activity in normal lung cells.

This is the first study to report the synthesis of a deuterated derivative of DSePA (D-DSePA). More specifically, the invention demonstrates the higher therapeutic index of D-DSePA as compared to un-deuterated DSePA using the cellular models of human non-cancerous lung fibroblast cells (WI-38 and WI-26 VA4) and human NSCLC (A549, H1975 and H3122). Moreover, D-DSePA also potentiates radiation kill in cellular models of human NSCLC (A549) through additive effect while protecting non-cancerous human lung fibroblast (WI-38) cells from radiation kill. Discovery of the anticancer and radioprotective activities of D-DSePA with higher therapeutic index is a novel invention in context of its probable application in cancer treatment and in reducing radiotherapy side effects.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although method/process and materials similar or equivalent to those described herein can be used in the practice of the disclosed methods, the exemplary methods, devices and materials are described herein. It is to be understood that this disclosure is not limited to particular methods, and experimental conditions described, as such methods and conditions may vary. Hereinafter, the present invention will be described in detail with specific examples. However, this is not limited to the following examples.

### Example 1

### Method of Synthesis of D-DSePA

Solid sodium borohydride (125 mg, 3.31 mmol) was slowly added in small portions to a suspension of selenium powder (245 mg, 3.10 mmol) in D₂O (25 ml) with stirring at room temperature. The orange-brown solution of Na₂Se₂ was formed after consumption of black selenium, and then the whole was heated at 60° C. for 30 minutes. The reaction mixture was cooled down to room temperature and 3-bromopropionic acid-D₄ (487 mg. 3.10 mmol) in D₂O (30 ml) was dropwise added to it with stirring which continued for overnight. The product was extracted with diethyl cther (3×10 ml), the solvent was evaporated in vacuo to give a pale-yellow solid which was washed with hexane (2×10 ml). The solid was further recrystallized from diethyl ether-hexane mixture to give an off white crystalline solid of the title compound (yield 201 mg, 42%), m.p. 128° C. Analysis Calculated for C₆H₂D₈O₄Se₂: C, 23.09; H, 0.65%. Found: C, 23.00; H, 3.12%.

General synthesis and characterization methods—All reactions were carried out under nitrogen atmosphere using conventional Schlenk techniques. Solvents were dried by standard methods with subsequent distillation under inert atmosphere. Selenium metal, 3-bromopropionic acid-D₄, D₂O and NaBH₄ were obtained from commercial sources. Elemental analyses were carried out on a Thermo Fischer Flash EA1112 CHNS elemental analyzer. Infrared spectra were recorded as KBr plates on a Jasco FT-IR 6100 spectroscopy. The mass spectra were recorded on a GC-MS QP 2010 Ultra (Shimadzu) mass spectrometer coupled with direct sample inlet device DI-MS 2010. The ¹H, ¹³C{¹H} and ⁷⁷Se{¹H} NMR spectra were recorded on a Bruker Avance-II spectrometer operating at 300, 75.5 and 57.2 MHz, respectively. Chemical shifts are relative to internal solvent peak for ¹H and ¹³C{¹H} NMR spectra and external Ph₂Se₂ (δ 463 ppm relative to Me₂Se) in CDCl₃ for ⁷⁷Sc{¹H} NMR spectra.

X-ray Crystallography—A Rigaku-Oxford make XtaLAB Synergy, Dualflex X-ray diffractometer was employed for crystal screening, unit cell determination, and data collection. The goniometer was controlled using the APEX3 software suite. The X-ray radiation employed, was generated from a Mo X-ray tube Kα (λ=0.71073 Å). All data were integrated with [unknown integration program] and a multi-scan absorption correction using SCALE3. ABSPACK was applied to correct the data for absorption effects. Systematic reflection conditions and statistical tests of the data suggested the space group Cc. The structure was solved by direct methods using SHELXS and refined by full-matrix least-squares methods against F2 by SHELXL-2017/1. Hydrogen atoms were placed in idealized positions and were set riding on the respective parent atoms. All non-hydrogen atoms were refined with anisotropic thermal parameters. ORTEP and Mercury were employed for the final data presentation and structure plots.

Cell culture and synthesis of DSePA—DSePA was synthesized and characterized as described in our previous reports. Human A549 cells (Adenocarcinoma; Non-small cell lung cancer), H1975 (Adenocarcinoma; Non-small cell lung cancer), H3122(Adenocarcinoma; Non-small cell lung cancer), WI-38 (human lung fibroblast) and WI-26 VA4 (human lung fibroblast) cells maintained at Bhabha Atomic Research Centre (BARC) were cultured in growth medium (DMEM/RPMI) supplemented with 10% FBS, 100 U/ml penicillin and 100 μg/ml streptomycin at 37° C. in a humidified 5% CO₂ environment. The stock solutions of D-DSePA and DSePA were prepared fresh in cell culture medium and diluted with the same to achieve the desired concentrations.

MTT Assay—To evaluate the anti-proliferative effect of D-DScPA and DScPA, Cells (˜5×10³) suspended in 100 μl of complete culture medium were seeded in each well of 96 well plates. The cells were allowed to attach and grow for 24 h, treated with desired concentrations of D-DSePA/DScPA dissolved in culture medium for 48 h and then processed for MTT assay as described in literature. The percentage (%) cytotoxicity was calculated from the decrease in absorbance at 570 nm of treated groups as compared to that of control group. The % cytotoxicity was plotted as a function of the concentration of D-DSePA and DSePA and from this curve, the concentration of D-DSePA/DScPA causing 50% toxicity (IC₅₀) was estimated. The therapeutic index was calculated by normalizing the IC₅₀ concentration of D-DSePA/DSePA in non-cancerous cells to that in cancerous cells.

Clonogenic assay—Clonogenic assay was performed according to the method reported previously. In brief, cells treated with drug (D-DScPA/DScPA) and/or irradiated were cultured for 7 days for the development of macroscopic colonies. After 7 days, colonies were fixed with absolute methanol for 5 min, stained with 0.5% crystal violet for 5 min, washed with distilled water to remove excess stain and counted manually under a stereo microscope. Control cells without irradiation or drug treatment were also processed in a similar manner to obtain the plating efficiency. A cluster of 25-50 blue-stained cells was considered as a colony. The plating efficiency and the survival fraction under irradiated and drug treated conditions were calculated using following formulae:

Plating Efficiency=[No. of colonies/No. of cells plated]×100

Survival fraction=No. of colonies/[No. of cells plated×(plating efficiency/100)]

The plating efficiency of A549 and WI38 cells was calculated by seeding ˜300 and ˜500 cells respectively. For determining anticancer activity, the cell number of A549 was fixed at ˜500 in all the treatment groups. For studying radioprotection and radio sensitization, the cell numbers of WI38 and A549 cells were varied, as cells tend to die with increasing absorbed dose.

Dose modification factor (DMF)—For this, survival fractions (SF) of the control and drug (D-DSePA/DSePA) treated cells were estimated at various absorbed doses of y-radiation ranging from 2 to 12 Gy by performing clonogenic assay. The data of survival fraction (log scale) against the radiation absorbed dose (D) (linear scale) was fitted with the quadratic dose response equation (SF=αD+βD²). From the fitted hyperbolic curve D₁₀ (the dose which decreased survival fraction from 1.0 to 0.1) values under drug treated (D₁₀(Drug+Radiation)) and untreated conditions (D₁₀(Radiation)) were estimated. From this DMF was calculated as (DMF=D₁₀(Drug+Radiation)/D₁₀(Radiation))

Data provided above shows a simple and scalable method comprising deuterated precursors to synthesize the D-DSePA with high yield efficiency. Further studies involving cellular models of human non-cancerous lung fibroblast (WI-38) and human NSCLC (A549, H1975 and H3122) established that D-DSePA has ˜7 folds higher therapeutic index as compared to un-deuterated DSePA. Additionally, D-DSePA also potentiates radiation kill in cellular model of human NSCLC (A549) while protecting non-cancerous lung fibroblast (WI-28) cells from radiation kill. Considering that DSePA has previously been shown to be effective through oral (daily) route of administration, the demonstration of its newer derivative with higher therapeutic index can further enhance the appeal in terms of patient compliance.

It will be appreciated by those skilled in the art that the present invention can be embodied in other specific forms without departing from the essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

What is claimed is:

1. A method of synthesis of a organodiselenide derivative 3,3′-diselenodipropionic acid-D₈ (D-DSePA), wherein the method comprises reacting sodium selenide (Na₂Se₂) and 3-bromopropionic acid-D₄ in D₂O solvent.

2. The method as claimed in claim 1, wherein the precursors are selenium powder, sodium borohydride and 3-bromopropionic acid-D₄.

3. The method as claimed in claim 1, wherein the reaction is carried out in deuterated water (D₂O).

4. A method of inhibiting growth/proliferation of non-small cell lung cancer (NSCLC) with higher therapeutic index comprising treating cellular models of NSCLC and non-cancerous lung fibroblast with deuterated 3,3′-diselenodipropionic acid (D-DSePA) in vitro.

5. The method as claimed in claim 4, wherein the NSCLC is a A549 or H1975 or H3122 cell type.

6. The method as claimed in claim 4, wherein the non-cancerous lung fibroblast is a WI-38 or WI-26 VA4 cell type.

7. A method of inhibiting the growth/proliferation of non-small cell lung cancer (NSCLC) comprising treating/incubating cellular models of NSCLC with γ-irradiation and deuterated 3,3′-diselenodipropionic acid (D-DSePA) in vitro.

8. The method as claimed in claim 7, wherein the NSCLC is a A549 cell type.

9. The method as claimed in claim 7, wherein cells are γ-irradiated in the absorbed dose range of 2-8 Gy.

10. The method as claimed in claim 7, wherein cells are treated with D-DSePA at a concentration of 0.5 μM.

11. The method as claimed in claim 7, wherein cells are treated with D-DSePA immediately after radiation exposure.

12. A method of radioprotection comprising treating/incubating non-cancerous cells with deuterated 3,3′-diselenodipropionic acid (D-DSePA) in vitro.

13. The method as claimed in claim 12, wherein the non-cancerous cell is WI-38 cell type.

14. The method as claimed in claim 12, wherein cells are irradiated in the absorbed dose range of 2-12 Gy.

15. The method as claimed in claim 12, wherein cells are treated with D-DSePA at a concentration of 0.5 μM.

16. The method as claimed in claim 12, wherein cells are treated with D-DSePA at 24 hours prior to radiation exposure.
